(19) United States
(12) Patent Application Publication
(10) Pub. No.: US 2011/0306069 A1
(43) Pub. Date: Dec. 15, 2011

(54) Corin As A Marker For Heart Failure

(76) Inventors: Qingyu Wu, Orange Village, OH (US); Shenghan Chen, Beachwood, OH (US); Edward Plow, Solon, OH (US); Ningzheng Dong, Suzhou (CN); Changgeng Ruan, Suzhou (CN)

(21) Appl. No.: 13/202,275

(22) PCT Filed: Feb. 19, 2010
(86) PCT No.: PCT/US2010/024744

§ 371 Date: Aug. 18, 2011

Related U.S. Application Data
(60) Provisional application No. 61/208,085, filed on Feb. 19, 2009.

(51) Int. Cl. G01N 33/535

(52) U.S. Cl. 435/7.92

(57) ABSTRACT

The invention is a method of assessing whether an individual is afflicted with congestive heart failure (CHF) comprising comparing the level of corin or portion thereof in the individual to the level of corin or portion thereof in a control, wherein a decrease in the level of corin or portion thereof in the individual compared to the level of corin or portion thereof in the control indicates that the individual is afflicted with CHF. The methods described herein can be also be used to determine the severity of CHF in an individual, whether an individual's treatment for CHF is effective and whether an individual is at risk of developing CHF.

This application claims the benefit of U.S. Provisional Application No. 61/208,085, filed on Feb. 19, 2009. The entire teachings of the above application(s) are incorporated herein by reference.

### BACKGROUND OF THE INVENTION

Congestive heart failure (CHF) is a major cardiovascular disease, afflicting more than 5 million patients in the U.S. alone (AHA. Heart disease and stroke statistics—2008 update. Dallas, Tex.: American Heart Association). When the disease progresses to the end stage, it becomes refractory to medical treatment. As a result, the disease has a very high mortality with one in five patients dying within a year. Thus, early diagnosis and medical intervention are important for managing this life-threatening disease.

Atrial and B-type natriuretic peptides (ANP and BNP) are cardiac hormones that act as a compensatory system in the setting of heart failure, and are elevated in CHF. BNP is used as a diagnostic biomarker for CHF. The development of the BNP-based tests has improved the diagnosis and disease management for CHF (Maisel, A., Circulation, 105(20):2328-2331 (2002)). These tests, however, have their limitations. For example, a commonly used BNP test failed to detect approximately 1 in 5 patients with established heart failure (Tang, W H., et al., Circulation, 108(24):2964-2966 (2003)). Moreover, the tests do not distinguish BNP from its precursor, pro-BNP, and therefore, the results may not reflect the biological activity of the peptide (Hawkridge, A M., et al., Proc. Natl. Acad. Sci., USA, 102(48):17442-17447 (2005); Liang, F., et al., J. Am. Coll. Cardiol., 49(10):1071-1078 (2007)). Clearly, better diagnostic tests are needed to improve the diagnostic accuracy for patients with CHF (Maisel, A., Circulation, 105(20):2328-2331 (2002)).

### SUMMARY OF THE INVENTION

Described herein are methods that were developed to measure corin in human plasma. Using the methods it was found that the levels of plasma corin were significantly reduced in patients with heart failure, and that the degree of the reduction correlated with the severity of the disease. Thus, described herein are methods for the diagnosis of heart failure using corin as a biomarker.

Accordingly, in one aspect, the invention is a method of assessing whether an individual is afflicted with congestive heart failure (CHF) comprising assessing the level of corin or portion thereof in the individual.

In a particular aspect, the invention is a method of assessing whether an individual is afflicted with congestive heart failure (CHF) comprising comparing the level of corin or portion thereof in the individual to the level of corin or portion thereof in a control, wherein a decrease in the level of corin or portion thereof in the individual compared to the level of corin or portion thereof in the control indicates that the individual is afflicted with CHF. In one embodiment, the level of corin in the individual is detected in a sample obtained from the individual such as a body fluid.

In another aspect, the invention is a method of assessing whether an individual is afflicted with congestive heart failure (CHF) comprising detecting the level of corin or portion thereof in the individual and comparing the level of corin or portion thereof in the individual to the level of corin or portion thereof in a control. A decrease in the level of corin or portion thereof in the individual compared to the level of corin or portion thereof in the control indicates that the individual is afflicted with CHF. The method can further comprise obtaining a sample from the individual and detecting the level of corin or portion thereof in the sample obtained from the individual.

In another aspect, the invention is a method of determining the severity of congestive heart failure (CHF) in an individual in need thereof comprising comparing the level of corin or portion thereof in a sample of the individual to the level of corin or portion thereof in a control, wherein the greater the decrease in the level of corin or portion thereof in the sample compared to the level of corin or portion thereof in the control, the greater the severity of CHF in the individual. The severity of the CHF can be, for example, stage II CHF, stage III CHF or stage IV CHF.

In another aspect, the invention is a method of assessing whether an individual's treatment for congestive heart failure (CHF) is effective comprising comparing the level of corin or portion thereof in a sample of the individual during treatment, after treatment or a combination thereof to the level of corin or portion thereof in a sample of the individual prior to treatment, wherein an increase in the level of corin or portion thereof in the sample of the individual during or after treatment compared to the level of corin or portion thereof in the sample of the individual prior to treatment is an indication that the individual's treatment for CHF is effective.

In another aspect, the invention is a method of assessing whether an individual is at risk of developing congestive heart failure (CHF) comprising comparing the level of corin or portion thereof in a sample of the individual to the level of corin or portion thereof in a control, or a baseline level of corin or portion thereof in the individual, wherein a decrease in the level of corin or portion thereof in the sample compared to the level of corin or portion thereof in the control or in the baseline level, is an indication that the individual is at risk of developing CHF.

### BRIEF DESCRIPTION OF THE DRAWINGS

FIG. 1 is a schematic of the corin protein domain structure. In the schematic of the corin protein domain structure, the transmembrane domain (TM), frizzled-like cysteine-rich domains (Fz1, Fz2), LDL receptor repeats (LDLR), scavenger receptor cysteine-rich repeat (SR) and protease domain (Protease) with active site residues histidine (H), aspartate (D) and serine (S) are indicated.

FIG. 2 is a graph showing plasma corin levels in normal controls and patients with congestive heart failure (CHF) and acute myocardial infarction (AMI).

FIG. 3 is a graph of plasma corin levels in normal controls and patients with CHF (NYHA Classes II to IV); * p<0.05, ** p<0.001 vs. control.

FIGS. 4A-4B is the nucleotide sequence of human corin (SEQ ID NO: 1).

FIG. 5 is the amino acid sequence of human corin (SEQ ID NO: 2).

FIGS. 6A-6B is the nucleotide sequence of mouse corin (SEQ ID NO: 3).

FIG. 7 is the amino acid sequence of mouse corin (SEQ ID NO: 4).

### DETAILED DESCRIPTION OF THE INVENTION

Corin is an enzyme present on the surface of heart cells (Yan, W., et al., J. Biol. Chem., 274(21):14926-14935 (1999)). Corin converts pro-ANP and pro-BNP to active ANP and BNP, which is essential for maintaining normal blood pressure and cardiac function (Yan, W., et. al., Proc. Natl. Acad. Sci., USA, 97(15):8525-8529 (2000)). In mice, lack of corin causes hypertension and cardiac hypertrophy (Chan, J C., et al., proc. natl. Acad. Sci, USA, 102(3):785-790 (2005)). In humans, corin gene variants are associated with hypertension and cardiac hypertrophy in African-Americans (Dries, D L, et al., Circulation, 112(16):2403-2410 (2005)), a population known for its high prevalence of hypertension and heart disease. At the 2008 American Heart Association (AHA) annual meeting it was reported that lower plasma corin levels were associated with a higher incidence of major adverse cardiovascular events post percutaneous coronary intervention (Circulation. 2008; 118:S1084).

Corin normally exists on heart muscle cell surface, and can be released into the circulation. Described herein is the development of an ELISA-based assay which measured plasma corin levels in patients with congestive heart failure (CHF). Using the assay, it was shown that plasma corin levels can be used as a biomarker for the diagnosis of heart disease such as CHF. The data provided herein shows that corin protein was detected in human plasma and that its levels were significantly lower in patients with CHF (431±311 vs. 716±315 μg/ml, p<0.0001). Importantly, the reduction of the corin levels directly correlated with the severity of the disease. In contrast, plasma corin levels were not significantly altered in patients with acute myocardial infarction (AMI). These data indicate that corin-based assays can be developed and used as specific diagnostic tests for CHF.

Accordingly, in one aspect, the invention is a method of assessing whether an individual is afflicted with congestive heart failure (CHF) comprising assessing the level of corin or portion thereof in the individual.

In a particular aspect, the invention is directed to a method of assessing whether an individual is afflicted with congestive heart failure (CHF) comprising comparing the level of corin or portion thereof in the individual to the level of corin or portion thereof in a control, wherein a decrease in the level of corin or portion thereof in the individual compared to the level of corin or portion thereof in the control indicates that the individual is afflicted with CHF.

In another aspect, the invention is directed to a method of assessing whether an individual is afflicted with congestive heart failure (CHF) comprising detecting the level of corin or portion thereof in the individual; and comparing the level of corin or portion thereof in the individual to the level of corin or portion thereof in a control, wherein a decrease in the level of corin or portion thereof in the individual compared to the level of corin or portion thereof in the control indicates that the individual is afflicted with CHF. The method can further comprise obtaining a sample from the individual prior and detecting the level of corn or portion thereof in the sample obtained from the individual.

In another aspect, the invention is a method of determining the severity of congestive heart failure (CHF) in an individual in need thereof comprising comparing the level of corin or portion thereof in a sample of the individual to the level of corin or portion thereof in a control, wherein the greater the decrease in the level of corn or portion thereof in the sample compared to the level of corin or portion thereof in the control, the greater the severity of CHF in the individual. The severity of the CHF can be, for example, stage II CHF, stage III CHF or stage IV CHF.

The discovery that reduction of plasma corn levels reflects the underlying pathology in the heart of CHF patients provides not only for the diagnosis of CHF but also for identifying individuals at risk for developing CHF and for monitoring patient responses to various medical treatments (e.g., surgical treatments such as coronary bypass surgery, angioplasty, insertion of a pacemaker; and medical treatments such as the use of diuretics, inotropes, digoxin, ACE inhibitors, vasodilators, nitrates, natriuretic peptides, natriuretic peptide receptor agonists, hydralazine, beta blockers, antihypertensives such as calcium channel blockers, angiotensin receptor blockers and endothelin receptor blockers).

Thus, another aspect of the invention is a method of assessing whether an individual's treatment for congestive heart failure (CHF) is effective comprising comparing the level of corin or portion thereof in a sample of the individual during treatment, after treatment or a combination thereof to the level of corin or portion thereof in a sample of the individual prior to treatment, wherein an increase in the level of corin or portion thereof in the sample of the individual during or after treatment compared to the level of corin or portion thereof in the sample of the individual prior to treatment is an indication that the individual's treatment for CHF is effective.

In yet another aspect, the invention is a method of assessing whether an individual is at risk of developing congestive heart failure (CHF) comprising comparing the level of corin or portion thereof in a sample of the individual to the level of corin or portion thereof in a control, or to a baseline level in the individual's sample, wherein a decrease in the level of corin or portion thereof in the sample compared to the level of corin or portion thereof in the control or the baseline sample is an indication that the individual is at risk of developing CHF. As appreciated by those of skill in the art, a “baseline level” of corin refers to the level of corin in the individual at the beginning of the assessment (the initial assessment) from which any variation in the level of corin in the individual can be measured. As also appreciated by those of skill in the art, in the method of assessing whether an individual is at risk of developing CHF can be performed on the individual over a period of time (e.g., days, months, years).

Heart failure, also called congestive heart failure, is a life-threatening condition in which the heart can no longer pump enough blood to the rest of the body. Typically, heart failure is a chronic, long-term condition, although it can sometimes develop suddenly, and can affect the right side, the left side, or both sides of the heart. As the heart's pumping action is lost, blood can back up into other areas of the body, including the gastrointestinal tract, arms, and legs (right-sided heart failure), liver, and lungs (left-sided heart failure). Heart failure results in a lack of oxygen and nutrition to organs, which damages them thereby reducing their ability to function properly. Most areas of the body can be affected when both sides of the heart fail. The most common causes of heart failure are coronary artery disease and high blood pressure. Other structural or functional causes of heart failure include cardiomyopathy, dilated cardiomyopathy, hypertrophic cardiomyopathy, restrictive cardiomyopathy, congenital heart disease, heart valve disease, heart tumor, and lung disease. Heart failure becomes more common with advancing age. Increased risk for developing heart failure can occur in individuals who are overweight, have diabetes, smoke cigarettes, abuse alcohol, or use drugs such as cocaine.

Symptoms of heart failure are usually graded based on a special classification system developed by, for example, the New York Heart Association (NHYA) classification (NYHA Class I, II, III or IV) or the American College of Cardiology/American heart Asociation (AHA) classification (AHA Stage A, B, C or D). The classifications are based on symptoms experienced during physical activity.

In the NHYA classification, Class I patients have asymptomatic heart failure, meaning they do not show symptoms; Class II patients have mild heart failure; Class III patients have moderate to severe heart failure, and; Class IV patients have severe heart failure. More specifically, Class I patients have no limitation of activities, they suffer no symptoms from ordinary activities; Class II patients have slight, mild limitation of activity, they are comfortable with rest or with mild exertion; Class III patients have marked limitation of activity, they are comfortable only at rest; and Class IV patients should be at complete rest, confined to bed or chair, any physical activity brings on discomfort and symptoms occur at rest.

In the American College of Cardiology/American heart Association (AHA) classification, Stage A patients are at high risk for developing HF in the future but no functional or structural heart disorder; Stage B patients have a structural heart disorder but no symptoms at any stage; Stage C patients have previous or current symptoms of heart failure in the context of an underlying structural heart problem, but managed with medical treatment; Stage D patients have advanced disease requiring hospital-based support, a heart transplant or palliative care.

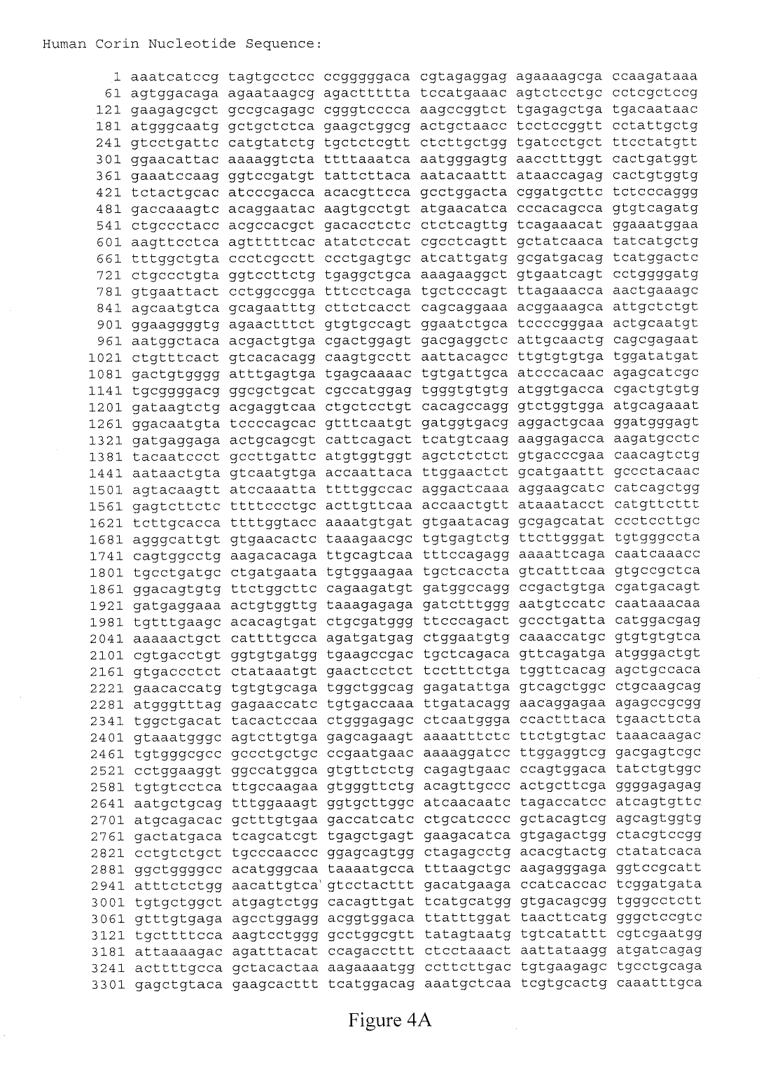

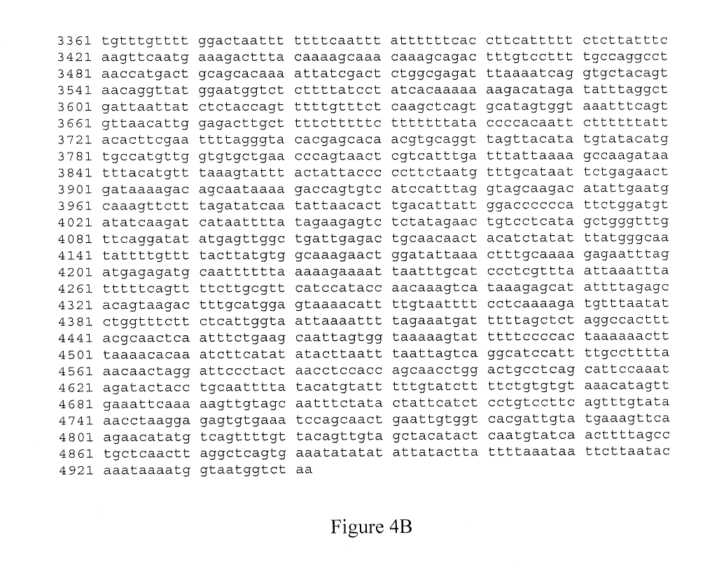

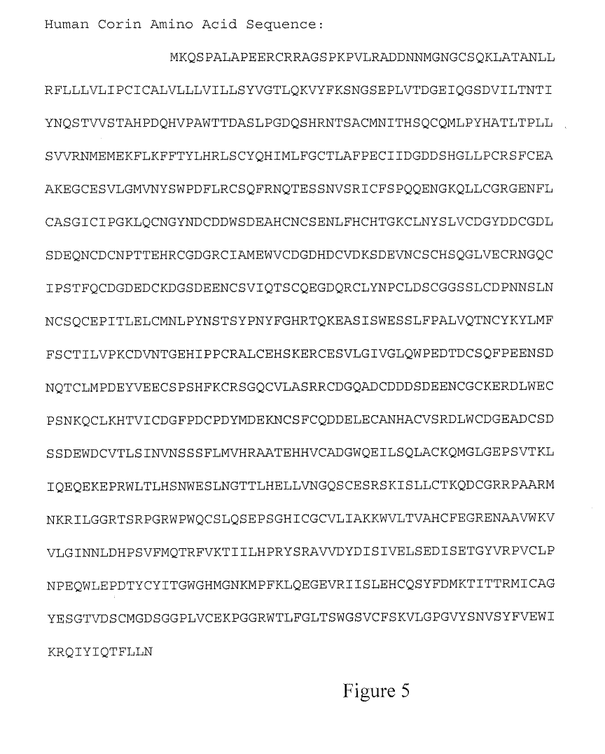

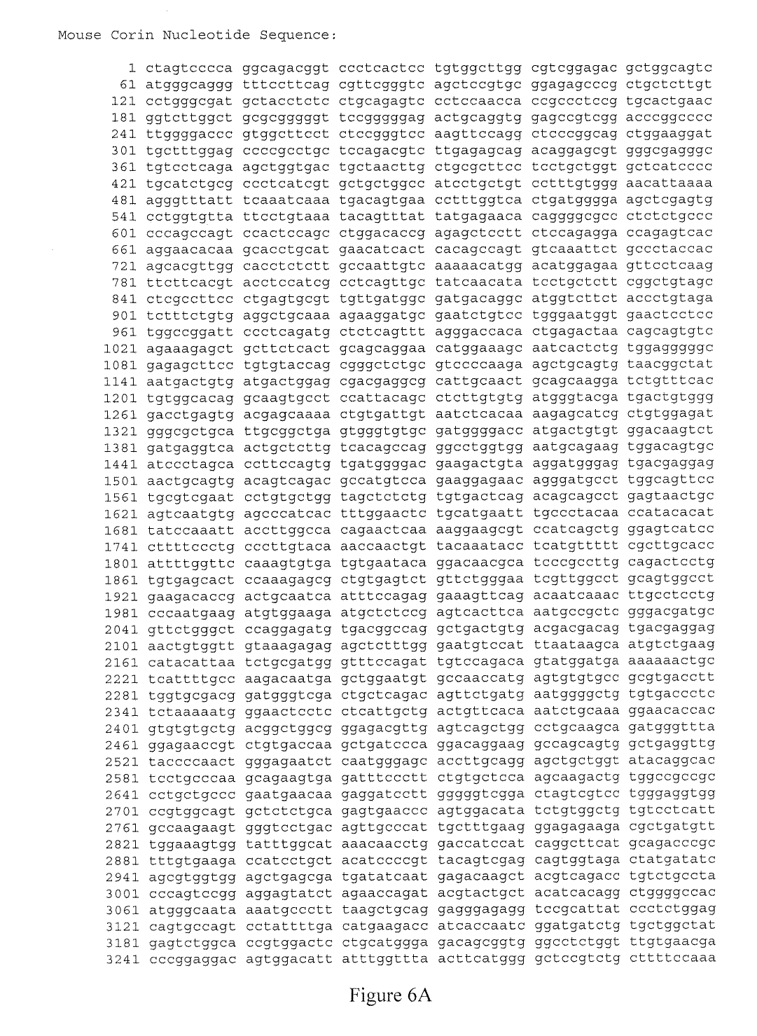

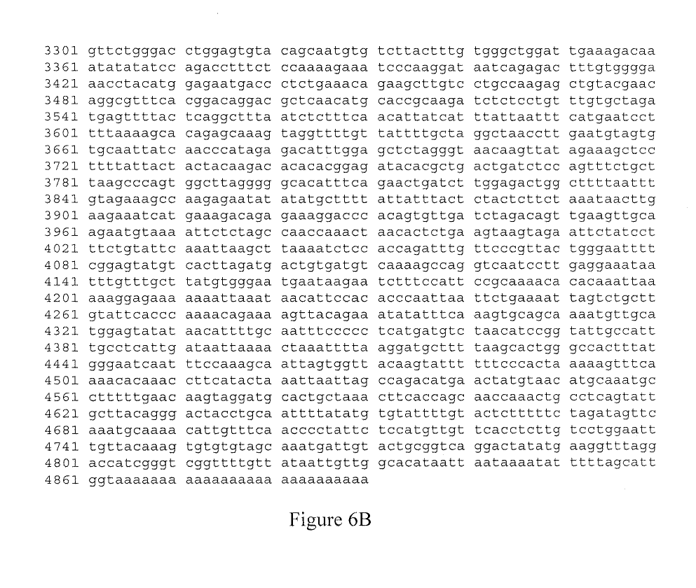

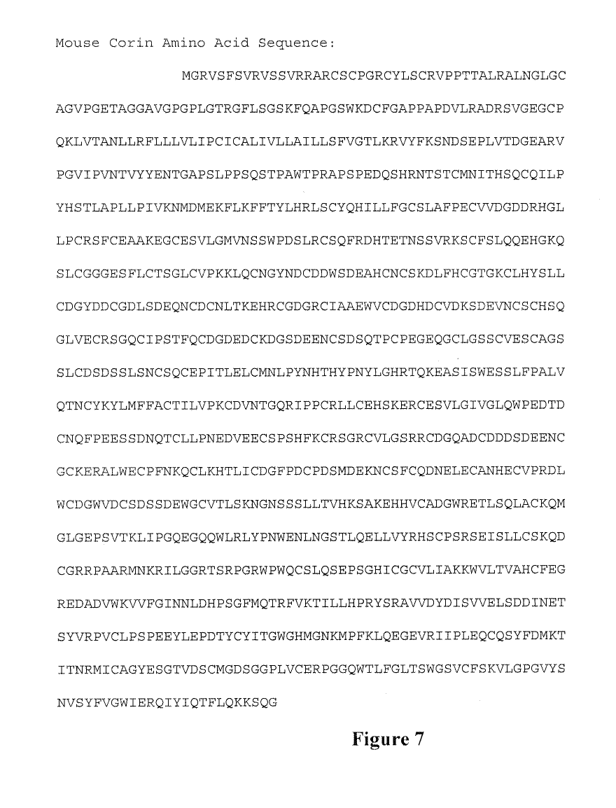

As used herein “corin” refers to a cardiac serine protease found in mammals. In the methods described herein, naturally occurring and engineered (e.g., recombinantly produced) variants of corin can also be detected. Human corin was cloned from the heart (Yan, W., et al., J. Biol. Chem., 274(21):14926-14935 (1999)). FIGS. 4A-4B show the nucleotide sequence of human corin (SEQ ID NO: 1) and FIG. 5 shows the amino acid sequence of human corin (SEQ ID NO:2). Mouse corin has also been cloned (Tomita, Y., et al. J. Biochem. (Tokyo), 124:784-789 (1998)). FIGS. 6A-6B show the nucleotide sequence of mouse corin (SEQ ID NO: 3) and FIG. 7 shows the amino acid sequence of mouse corin (SEQ ID NO: 4).

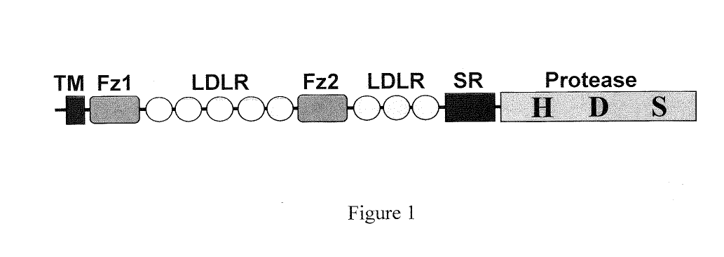

Human corin protein consists of 1042 amino acids and has an integral transmembrane domain near its N-terminus. In its extracellular region, there are two frizzled-like domains, eight LDL receptor repeats, a scavenger receptor-like domain, and a trypsin-like protease domain (FIG. 1). Specifically, corin is a type II transmembrane serine protease of the trypsin superfamily having the following structurally distinct domains: a transmembrane/signal peptide, frizzled domains, low density lipoprotein receptor repeats (LDLR), scavenger receptor cysteine-rich repeats (SRCR) and a serine protease catalytic domain. Human corin is comprised of 1042 amino acids (SEQ ID NO: 2) which include a cytoplasmic tail at its N-terminus (amino acids 1 to 45 of SEQ ID NO: 2) followed by a transmembrane domain (amino acids 46 to 66 of SEQ ID NO: 2), a stem region composed of two frizzled-like cysteine-rich domains (CRD, amino acids 134 to 259 and 450 to 573 of SEQ ID NO: 2), eight low density lipoprotein receptor repeats (LDLR, amino acids 268 to 415 and 579 to 690 of SEQ ID NO: 2), a macrophage receptor-like domain (SRCR, amino acids 713 to 800 of SEQ ID NO: 2) and a serine protease catalytic domain at its C-terminus (CAT, amino acids 802 to 1042 of SEQ ID NO: 2). Amino acids 801 through 805 of SEQ ID NO: 2 (i.e., ArgIleLeuGlyGly or RILGG) is a conserved activation cleavage site, in which proteolytic cleavage of the peptide bind between Arg801 and Ile802 generates a catalytically active corin. See U.S. Pat. No. 6,806,075; U.S. Pat. No. 7,176,013; PCT Published Application No. WO 03/102135; and Wu, Q, Frontiers in Bioscience, 12:4179-4190 (2007) all of which are incorporated herein by reference.

Corin is made primarily in cardiomyocytes. Corin mRNA expression appeared to be higher in the atrium than the ventricle. Corin mRNA also was found in scar myofibroblasts in rat hearts and in pregnant uterus. Lower levels of corin mRNA were detected in other tissues including developing kidneys and bones. In contrast, corin mRNA was not found in other muscle-rich tissues such as stomach, small intestine, bladder, skeletal muscle, and non-pregnant uterus.

Functional studies show that corin converts pro-ANP and pro-BNP to active ANP and BNP (Yan, W., et al., Proc. Natl. Acad. Sci., USA, 97(15):8525-8529 (2000)), cardiac hormones that regulate blood pressure and salt-water balance. In mice, corin deficiency caused hypertension that is salt sensitive and exacerbated during pregnancy (Chan, J C., et al., Proc. Natl. Acad. Sci., USA, 102(3):785-790 (2005)). Corin knockout mice also had cardiac hypertrophy and reduced cardiac function. Recently, nonsynonymous single nucleotide polymorphisms (SNPs) are found in the human corin gene. A minor allele with these SNPs is more common in African Americans than Caucasians and is associated with hypertension and left ventricular hypertrophy. Amino acid changes caused by these SNPs prevented corin zymogen activation and impaired its natriuretic peptide processing activity (Wang, W., et al., Circ. Res., 103 (5):502-508 (2008)). Together these data indicate that corin is essential in maintain normal blood pressure and cardiac function in vivo and that corin deficiency may contribute to hypertension and heart disease in patients.

As will be appreciated by those of skill in the art, in the methods described herein, corn can be detected, directly or indirectly, using a variety of methods known in the art. For example, corin nucleic acid or portion thereof, corn protein or portion thereof, corn activity and combinations thereof can be detected using a variety of appropriate methods, including for example, methods for detecting the quantity of mRNA transcribed from the corin gene, the quantity of cDNA produced from the reverse transcription of the mRNA transcribed from the corin gene, the quantity of the corn polypeptide or protein encoded by the corn gene, or the activity of the corn polypeptide or protein encoded by the corin gene. In particular embodiments, the corin is a mammalian corn such as a primate (e.g., human) corn, a murine (e.g., mouse, rat) corin, a feline corn, a canine corin, a bovine corin and the like.

Such methods can be performed on a sample by sample basis or modified for high throughput analysis. Samples used for this invention encompass body fluid, solid tissue samples, tissue cultures or cells derived therefrom and the progeny thereof and sections or smears prepared from any of these sources or any other samples that may contain a cell having a corin gene described herein.

In assaying for corin polypeptide, a corn polypeptide (also referred to herein as a protein) or a portion thereof can be detected. As will be appreciated by those of skill in the art, the portion of corin that can be detected in the methods can be a portion of corin which has corin activity (e.g., a biologically active portion or corin) or can be a portion of corin which does not have corin activity. In one aspect, the portion of corin detected in the methods is a biologically active portion of a corin poplypeptide. In other aspects, the corin polypeptide or biologically active portion thereof is a mammalian corin polypeptide such as a primate (e.g., human) corin, a murine (e.g., mouse, rat) corin, a feline corin, a canine corin, a bovine corin and the like. In a particular embodiment, the polypeptide is all or a biologically active portion of SEQ ID NO:2.

As used herein, a “biologically active portion of a corin polypeptide” is a portion of a corin polypeptide that retains one or more functions/activities of corin. Functions of corin include conversion of proANP to active ANP and conversion of pro-BNP to active BNP. Corin may also be able to cleave and thus activate or inactivate other biological peptides in a cell or individual. Examples of a biologically active portion of a corin polypeptide comprises at least one frizzled domain of the corin protein, at least one low density lipoprotein receptor (LDLR) repeat of the corin protein, a serine protease catalytic domain of the corin protein or a combination thereof. In one embodiment, the biologically active portion is a soluble corin polypeptide that lacks all, or substantially all, of the transmembrane domain. For example, a soluble corin polypeptide can include all or a portion of the extracellular domain. In one embodiment, the biologically active portion of the mammalian corin protein is a soluble corin polypeptide comprising all or a portion of the corin extracellular domain (e.g., from about amino acid 67 to about amino acid 1042 of SEQ ID NO: 2) (e.g., see U.S. Pat. No. 6,806,075 which is incorporated herein by reference). In another embodiment, the biologically active portion comprises a serine protease catalytic domain of a corin polypeptide. In yet another embodiment, the biologically active portion of the mammalian corin protein is a soluble corin polypeptide comprising the serine protease catalytic domain (e.g., from about amino acid 802 to about amino acid 1042 of SEQ ID NO: 2 (see U.S. Pat. No. 6,806,075 which is incorporated herein by reference).

In assaying for a corin polypeptide or portion thereof, a variety of techniques are available in the art. They include but are not limited to radioimmunoassays, ELISA (Enzyme Linked Immunoradiometric Assays), “sandwich” immunoassays, immunoradiometric assays, in situ immunoassays (using e.g., colloidal gold, enzyme or radioisotope labels), western blot analysis, immunoprecipitation assays, immunofluorescent assays, PAGE-SDS and protein chips. One means to determine corin protein level involves (a) providing a biological sample containing corin polypeptide(s); and (b) measuring the amount of any immunospecific binding that occurs between an antibody reactive to the corin polypeptide or portion thereof and corin polypeptide(s) in the sample, in which the amount of immunospecific binding indicates the level of the corin polypeptide(s).

Antibodies that specifically recognize and bind to a corin polypeptide or portion thereof are used in immunoassays. Such antibodies may be purchased from commercial vendors (R&D Systems, Abcam, and Santa Cruz Biotechnology) or generated and screened using methods well known in the art. Alternatively, polyclonal or monoclonal antibodies that specifically recognize and bind the protein product of a gene of interest can be made and isolated using known methods. See, for example, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, N.Y. (jointly referred to herein as “Harlow and Lane”).

An antibody that is specific for corin or portion thereof is a molecule that selectively binds to corin but does not substantially bind to other molecules in a sample, e.g., in a biological sample that contains corin. The term “antibody,” as used herein, refers to an immunoglobulin or a part thereof, and encompasses any polypeptide comprising an antigen-binding site regardless of the source, method of production, and other characteristics. The term includes but is not limited to polyclonal, monoclonal, monospecific, polyspecific, humanized, human, single-chain, chimeric, synthetic, recombinant, hybrid, mutated, conjugated and CDR-grafted antibodies. The term “antigen-binding site” refers to the part of an antibody molecule that comprises the area specifically binding to or complementary to, a part or all of an antigen. An antigen-binding site may comprise an antibody light chain variable region (VL) and an antibody heavy chain variable region (VH). An antigen-binding site may be provided by one or more antibody variable domains (e.g., an Fd antibody fragment consisting of a VH domain, an Fv antibody fragment consisting of a VH domain and a VL domain, or an scFv antibody fragment consisting of a VH domain and a VL domain joined by a linker). The term “anti-corin antibody,” or “antibody against corin,” refers to any antibody that specifically binds to at least one epitope of corin. As used herein, the term “selectively binds to” or “binding specificity” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. In particular aspects, the invention is directed to an antibody that has binding specificity (e.g., epitopic specificity) for all or a portion of a corin polypeptide.

The various antibodies and portions thereof can be produced using known techniques (Kohler and Milstein, Nature 256:495-497 (1975); Current Protocols in Immunology, Coligan et al., (eds.) John Wiley & Sons, Inc., New York, N.Y. (1994); Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 Bl; Queen et al., European Patent No. 0 451 216 B1; and Padlan, E. A. et al., EP 0 519 596 A1; Newman, R. et al., BioTechnology, 10: 1455-1460 (1992); Ladner et al., U.S. Pat. No. 4,946,778; Bird, R. E. et al., Science, 242: 423-426 (1988)).

As noted above, antibodies useful in the present invention can include polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Alternatively, functional equivalents of whole antibodies, such as antigen binding fragments in which one or more antibody domains are truncated or absent (e.g., Fv, Fab, Fab′, or F(ab)2 fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Genetically engineered antibodies include those produced by standard recombinant DNA techniques involving the manipulation and re-expression of DNA encoding antibody variable and/or constant regions. Particular examples include, chimeric antibodies, where the VH and/or VL domains of the antibody come from a different source to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Constructions of chimeric and CDR-grafted antibodies are described, for example, in European Patent Applications: EP-A 0194276, EP-A 0239400, EP-A 0451216 and EP-A 0460617. Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

In addition, or in the alternative, corin nucleic acid or portion thereof can be detected in the methods of the invention. In assaying for corin nucleic acid or portion thereof, nucleic acid contained in the individual (e.g., a sample of the individual) is first extracted according to standard methods in the art. For instance, nucleic acid can be isolated using various lytic enzymes or chemical solutions according to the procedures set forth, for example, in Methods of Enzymology, Vol. 194, Guthrie et al., eds., Cold Spring Harbor Laboratory Press (1990); Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001. The mRNA of a gene contained in the extracted nucleic acid sample is then detected by hybridization (e.g., Northern blot analysis) and/or amplification procedures according to methods widely known in the art or based on the methods exemplified herein (e.g., PCR: The Polymerase Chain Reaction, (Mullis et al., eds., 1994)).

Nucleic acid molecules exhibiting sequence complementarity or homology to a corin polynucleotide or portion thereof are useful as hybridization probes. It is known in the art that a “perfectly matched” probe is not needed for a specific hybridization. Minor changes in probe sequence achieved by substitution, deletion or insertion of a small number of bases do not affect the hybridization specificity. These probes can be used in radioassays (e.g., Southern and Northern blot analysis) to detect corin nucleic acid. In one aspect, nucleotide probes having complementary sequences over stretches greater than about 10 nucleotides in length are used, so as to increase stability and selectivity of the hybrid and, thereby, improving the specificity of particular hybrid molecules obtained. Alternatively, one can design nucleic acid molecules having gene-complementary stretches of more than about 25 or alternatively more than about 50 nucleotides in length or even longer where desired. Such fragments may be readily prepared by, for example, directly synthesizing the fragment by chemical means, by application of nucleic acid reproduction technology, such as the PCR™ technology with two priming oligonucleotides as described in U.S. Pat. No. 4,603,102 or by introducing selected sequences into recombinant vectors for recombinant production. In one aspect, a probe is about 50 to about 75, nucleotides or, alternatively, about 50 to about 100 nucleotides in length. These probes can be designed from the sequence of full length genes. In certain embodiments, it will be advantageous to employ nucleic acid sequences as described herein in combination with an appropriate means, such as a label, for detecting hybridization and therefore complementary sequences. A wide variety of appropriate indicator means are known in the art, including fluorescent, radioactive, enzymatic or other ligands, such as avidin/biotin, which are capable of giving a detectable signal. One can employ a fluorescent label or an enzyme tag, such as urease, alkaline phosphatase or peroxidase, instead of radioactive or other environmental undesirable reagents. In the case of enzyme tags, colorimetric indicator substrates are known which can be employed to provide a means visible to the human eye or spectrophotometrically, to identify specific hybridization with complementary nucleic acid-containing samples.

Hybridization reactions can be performed under conditions of different “stringency”. Relevant conditions include temperature, ionic strength, time of incubation, the presence of additional solutes in the reaction mixture such as formamide and the washing procedure. Higher stringency conditions are those conditions, such as higher temperature and lower sodium ion concentration, which require higher minimum complementarity between hybridizing elements for a stable hybridization complex to form. Conditions that increase the stringency of a hybridization reaction are widely known and published in the art. See, for example, Sambrook et al. supra.

Known amplification methods include PCR, MacPherson et al., PCR: A PRACTICAL APPROACH, (IRL Press at Oxford University Press (1991)). However, PCR conditions used for each application reaction are empirically determined. A number of parameters influence the success of a reaction. Among them are annealing temperature and time, extension time, Mg²⁺ ATP concentration, pH and the relative concentration of primers, templates and deoxyribonucleotides.

After amplification, the resulting DNA fragments can be detected by agarose gel electrophoresis followed by visualization with ethidium bromide staining and ultraviolet illumination. A specific amplification of differentially expressed genes of interest can be verified by demonstrating that the amplified DNA fragment has the predicted size, exhibits the predicated restriction digestion pattern and/or hybridizes to the correct cloned DNA sequence.

The probes also can be attached to a solid support for use in high throughput screening assays using methods known in the art. PCT WO 97/10365 and U.S. Pat. Nos. 5,405,783; 5,412,087 and 5,445,934; for example, disclose the construction of high density oligonucleotide chips which can contain one or more of the sequences disclosed herein. Using the methods disclosed in U.S. Pat. Nos. 5,405,783; 5,412,087 and 5,445,934; the probes of this invention are synthesized on a derivatized glass surface. Photoprotected nucleoside phosphoramidites are coupled to the glass surface, selectively deprotected by photolysis through a photolithographic mask and reacted with a second protected nucleoside phosphoramidite. The coupling/deprotection process is repeated until the desired probe is complete.

The expression level of a gene can also be determined through exposure of a nucleic acid sample to a probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See, U.S. Pat. Nos. 5,578,832 and 5,631,734. The obtained measurement is directly correlated with gene expression level.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein and the like), radiolabels (e.g., 3H, 125I, ³⁵S, ¹⁴C or ³²P) enzymes (e.g., horseradish peroxidase, alkaline phosphatase and others commonly used in an ELISA) and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate and colorimetric labels are detected by simply visualizing the colored label.

In addition, or in the alternative, corin activity can be detected in the methods of the invention. In assaying for corin activity a variety of methods known to those of skill in the art are also available for use in the methods of the invention (e.g., Wu, Q., Frontiers in Bioscience, 12:4179-4190 (2007); Yan, W., et al., Proc. Natl. Acad. Sci, USA, 97:8525-8529 (2000); Knappe, S. F., et al., J. Biol. Chem., 279:34464-34471 (2004); Knappe, S. F., J. Biol. Chem., 278:52363-52370 (2003); U.S. Pat. No. 6,806,075; U.S. Pat. No. 7, 176,013; PCT Published Application No. WO 03/102135, all of which are incorporated herein by reference in their entirety). For example, corin enzymatic activity can be detected using chromogenic or fluorogenic substrate-based assays (Knappe, S. F., J. Biol. Chem., 278:52363-52370 (2003)). In addition, pro-atrial natriuretic peptide-based assays to detect corin activity in a biological fluid such as plasma can be used (e.g., U.S. Pat. No. 6,806,075; U.S. Pat. No. 7,176,013).

In assessing whether an individual is afflicted with CHF, one typically conducts a comparative analysis of the subject and appropriate controls. Thus in one aspect of the invention, the method comprises comparing the level of corin or portion thereof in the individual with a control sample. As will be appreciated by those of skill in the art, the control sample can be an actual sample or it can be a value or range of values (e.g., from a control population) previously determined. For example, the control sample can be derived from a subject that lacks the clinical characteristics of CHF, referred to herein as a “normal control” or “negative control”. Examples of such a control includes one or more samples from one or more healthy individuals, a reference standard (e.g., using purified recombinant or native corn (e.g., human corin)) or a combination thereof. A lack of correlation between the subject and the negative control indicates that the individual is afflicted with CHF.

In the alternative, or in addition, the method can also include a control sample derived from a subject (hereinafter “positive control”), that exhibits CHF. A positive correlation between the subject and the positive control indicates that the individual is afflicted with CHF.

As used herein an “individual” refers to any subject in need of screening. In particular embodiments, the individual is a mammal, such as a primate (e.g., human), cow, sheep, goat, horse, dog, cat, rabbit, guinea pig, rat, mouse or other bovine, ovine, equine, canine feline, rodent or murine species). In one embodiment, the individual is a human.

Although the invention is performed herein using plasma samples so as to demonstrate that the method is non-invasive, rapid and easy to perform, one of skill in the art will appreciate that any suitable biological sample obtained from an individual can be used in the methods of the invention. Examples of suitable samples include biological fluids and tissue samples. Examples of a biological fluid that can be used in the methods include blood, plasma, urine and the like. Examples of a tissue sample include a tissue smear, a tissue scrape, and the like. Typically the sample is a biological fluid. In another embodiment, the sample comprises cells prepared from a subject's heart tissue.

The invention also provides an article of manufacture, also referred to herein as a kit, for use in diagnosis of heart failure. In one embodiment, the article of manufacture comprises a composition that detects corin (e.g., corin nucleic acid or portion thereof; corin polypeptide or portion thereof; corin activity; and combinations thereof). Such compositions include a probe and or primer that detects corin nucleic acid or portion thereof; an antibody that has binding specificity for corin or portion thereof; reagents that detect corin activity. The article of manufacture can also include manufacturer's instructions for use and packaging material.

The methods described herein can be used alone or together with current clinical tests for better diagnosis of CHF, and can likely also be used in the diagnosis of other cardiovascular diseases. The methods described herein have advantages in terms of sensitivity and specificity. Currently, several tests are used to measure plasma BNP and its derivatives for the diagnosis of CHF. Such tests include the Triage BNP (Biosite) which is based on antibodies against the C-terminal BNP, and N-terminal (NT)-pro-BNP (Roche Diagnostics and Siemens) which is based on antibodies against the N-terminal pro-BNP. These tests have their intrinsic problems because the antibodies used in these assays do not distinguish pro-BNP from BNP. For example, antibodies bind to BNP may also recognize pro-BNP and, as a result, it is not clear whether pro-BNP, BNP, or both are measured in these tests (Hawkridge, A M, et al., Proc. Natl. Acad. Sci., USA, 102(48):17442-17447 (2005); Liang, F., et al., J. Am. Coll. Cardiol., 49(10):1071-1078 (2007)). Thus, use of the methods described herein and these known tests will likely result in improved diagnosis of cardiovascular diseases such as CHF.

### Exemplification

### Soluble Corin as a Diagnostic for Heart Failure

### Methods

### ELISA

Human corin antigen in plasma samples was measured by an ELISA assay. Polyvinylchloride microtiter (96 wells) plates were coated with a polyclonal anti-human corin antibody (100 μL/well in a PBS solution containing 20 μg/mL of antibody). The plate was incubated overnight at room temperature, allowing the antibody to attach to the well. After washing with PBS to remove unbound antibody, a blocking solution (3% bovine serum albumin and 0.02% sodium azide in PBS) was added to the plate and incubated at room temperature for at least 1 hr to saturate non-specific binding sites. Following three additional washes with PBS, 100 μl of plasma samples was added to each well and incubated 2 h at room temperature. After three washes with PBS, 100 μl of a biotinylated anti-human corin antibody was added to each well and incubated 2 h at room temperature. Washing three times with PBS again, 100 μl of streptavidin-horseradish peroxidase was added to each well and incubated for 20 min at room temperature. Afterward, 100 μl of a horseradish peroxidase substrate (3,3′,5,5′-tetramethylbenzidine, TMB) was added to the plate and incubated for 20 min at room temperature. Next, 50 μl of 2N H₂SO₄ was added to stop the reaction. The optical density of each well was determined immediately using a SpectraMax M2/M2^{e} microplate reader (Molecular devices Corporation, Sunnyvale, Calif.) set to a wavelength of 450 nm. The concentration of soluble corin in plasma was calculated based on a standard curve.

### Results

### Soluble Corin in the Blood.

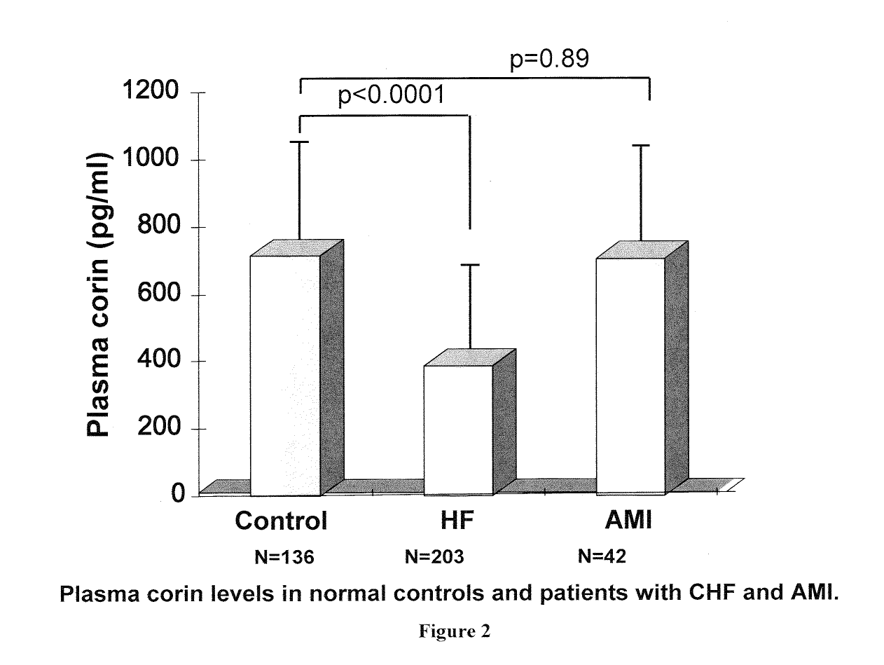

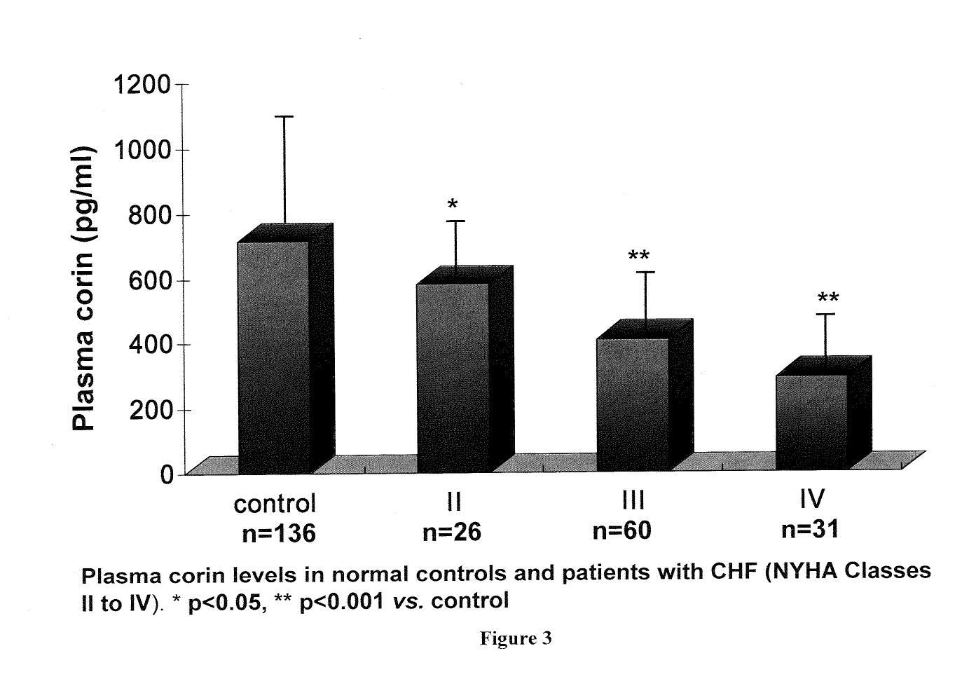

Topologically, corin belongs to the type II transmembrane serine protease family. Many enzymes from this family can be shed from the cell surface. As shown herein in cell culture, soluble corin was also detected in the culture medium, an indication of corin shedding under these conditions. Based on this showing, it was likely that similar shedding events could occur in vivo and that soluble corin could be detected in the blood. As described herein, to test this hypothesis an ELISA assay was developed and used to measure corin levels in human plasma. It was found that soluble corin indeed existed in the blood. In samples from healthy adults (n=136), plasma corin levels were 716±300 μg/mL (FIG. 2).

Plasma Corin Levels in Patients with Heart Disease

Next, plasma corin was measured in patients with CHF. As shown in FIG. 2, the plasma corin levels were significantly lower in patients with CHF. The degree of the reduction correlated with the severity of the disease (FIG. 2). Interestingly, plasma corin levels in patients with acute myocardial infarction (AMI) were similar to that of normal controls (FIG. 2), indicating that the reduction of plasma corin levels is closely related to CHF but not AMI. These data indicate that a corin-based assay can be used as a specific diagnostic test for CHF, and that plasma corin levels can also be a prognostic marker for CHF. Such a marker is useful in monitoring the response of CHF patients to the treatment.

### Conclusion

Provided herein is a soluble corin assay. Our data indicate that plasma corin concentrations were significantly lower in patients with CHF and inversely correlate with the severity of the disease. Current clinical tests measuring BNP and its derivatives have their limitations such as large assay variations and lack of sensitivity and specificity. Plasma corin levels can be used as an indicator for its expression and/or function in the heart. The data described herein shows that the lowest levels of plasma corin were found in a patient group with most severe (NYHA class IV) CHF. Thus, the reduction of plasma corin levels likely reflects the underlying pathology in the heart of CHF patients. Corin-based assay can be used not only for the diagnosis of CHF but also for monitoring patient responses to various medical treatments.

| Class IV Patients | Corin | BNP (Biosite) |
|---|---|---|
| 1 | 204 | 56 |
| 2 | 219 | 113 |
| 3 | 457 | 573 |
| 4 | 161 | 920 |
| 5 | 182 | 12 |
| 6 | 330 | 882 |
| 7 | 291 | 1490 |
| 8 | 48 | 914 |
| 9 | 32 | 33 |
| 10 | 78 | 1214 |
| 11 | 118 | 526 |
| 12 | 222 | 820 |
| 13 | 248 | 152 |
| 14 | 323 | 101 |
| 15 | 365 | 135.2 |
| 16 | 38 | 70 |
| 17 | 679 | 938 |
| 18 | 219 | 760 |
| 19 | 314 | 293 |
| 20 | 189 | 382 |
| 21 | 200 | 427 |
| 22 | 165 | 33 |

Normal corin levels: 714±274 pg/ml (Diagnostic cutoff: <440 pg/ml)

Plasma BNP levels for HF diagnosis: >100 pg/ml

False negative rate:
Corin 2/22 (9%)
BNP 5/22 (22.7%) (21.3% Tang, W H., et al., Circ. 108(24):2964-2966 (2003))
Reduction of false negative rate: 60%
No overlap in false negative sample

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of assessing whether an individual is afflicted with congestive heart failure (CHF) comprising detecting the level of corin or portion thereof in the individual and comparing the level of corin or portion thereof in the individual to the level of corin or portion thereof in a normal control, wherein a decrease in the level of corin or portion thereof in the individual compared to the level of corin or portion thereof in the control indicates that the individual is afflicted with CHF.

2. The method of claim 1 wherein the level of corin or portion thereof in the individual is detected in a sample obtained from the individual.

3. The method of claim 2 wherein the sample is a body fluid.

4. The method of claim 3 wherein the body fluid is blood, plasma or a combination thereof.

5. The method of claim 3 wherein the corin is soluble corin.

6. The method of claim 1 wherein the individual is human.

7. The method of claim 1 wherein the control comprises one or more samples from one or more healthy individuals, a reference standard or a combination thereof.

8. The method of claim 1 wherein the level of a corin polypeptide or portion thereof is detected.

9. The method of claim 8 wherein the corin polypeptide or portion thereof is detected using an antibody having binding specificity for all or a portion of corin.

10. The method of claim 9 wherein the antibody is a polyclonal antibody or a monoclonal antibody.

11. The method of claim 10 wherein the antibody has binding specificity for all or a portion of the extracellular domain of corin.

12. (canceled)

13. The method of claim 1 further comprising obtaining a sample from the individual prior to step a) and detecting the level of corin or portion thereof in the sample obtained from the individual.

14. (canceled)

15. (canceled)

16. (canceled)

17. (canceled)

18. (canceled)

19. (canceled)

20. (canceled)

21. (canceled)

22. (canceled)

23. A method of determining the severity of congestive heart failure (CHF) in an individual in need thereof comprising detecting the level of corin or portion thereof in the individual and comparing the level of corin or portion thereof in a sample of the individual to the level of corin or portion thereof in a normal control, wherein the greater the decrease in the level of corin or portion thereof in the sample compared to the level of corin or portion thereof in the control, the greater the severity of CHF in the individual.

24. The method of claim 23 wherein the severity of the CHF is stage II, stage III or stage IV.

25. The method of claim 23 wherein the level of corin or portion thereof in the individual is detected in a sample obtained from the individual.

26. The method of claim 25 wherein the sample is a body fluid.

27. The method of claim 26 wherein the body fluid is blood, plasma or a combination thereof

28. The method of claim 26 wherein the corin is soluble corin.

29. The method of claim 23 wherein the individual is human.

30. The method of claim 23 wherein the control comprises one or more samples from one or more healthy individuals, a reference standard or a combination thereof

31. The method of claim 23 wherein the level of a corin polypeptide or a portion thereof is detected.

32. The method of claim 31 wherein the corin polypeptide or portion thereof is detected using an antibody having binding specificity for corin.

33. The method of claim 32 wherein the antibody is a polyclonal antibody or a monoclonal antibody.

34. The method of claim 33 wherein the antibody has binding specificity for all or a portion of the extracellular domain of corin.

35. The method of claim 23 further comprising detecting the level of corin or portion thereof in the individual prior to comparing the level of corin or portion thereof in a sample of the individual to the level of corin or portion thereof in a control.

36. The method of claim 35 further comprising obtaining a sample from the individual prior to detecting the level of corin or portion thereof in the sample obtained from the individual.

37. A method of assessing whether an individual's treatment for congestive heart failure (CHF) is effective comprising detecting the level of corin or portion thereof in the individual and comparing the level of corin or portion thereof in a sample of the individual during treatment, after treatment or a combination thereof to the level of corin or portion thereof in a sample of the individual prior to treatment, wherein an increase in the level of corin or portion thereof in the sample of the individual during or after treatment compared to the level of corin or portion thereof in the sample of the individual prior to treatment is an indication that the individual's treatment for CHF is effective.

38. The method of claim 37 wherein the level of corin or portion thereof in the individual is detected in a sample obtained from the individual.

39. The method of claim 38 wherein the sample is a body fluid.

40. The method of claim 39 wherein the body fluid is blood, plasma or a combination thereof.

41. The method of claim 37 wherein the corin is soluble corin.

42. The method of claim 37 wherein the individual is human.

43. The method of claim 37 further comprising comparing the level of corin or portion thereof in a sample of the individual during treatment, after treatment or a combination thereof to the level of corin or portion thereof in a normal control.

44. The method of claim 37 wherein the level of a corin polypeptide or portion thereof is detected.

45. The method of claim 44 wherein the corin polypeptide or portion thereof is detected using an antibody having binding specificity for corin.

46. The method of claim 45 wherein the antibody is a polyclonal antibody or a monoclonal antibody.

47. The method of claim 46 wherein the antibody has binding specificity for all or a portion of the extracellular domain of corin.

48. The method of claim 37 further comprising detecting the level of corin or portion thereof in the individual prior to comparing the level of corin or portion thereof in a sample of the individual to the level of corin or portion thereof in a control.

49. The method of claim 48 further comprising obtaining a sample from the individual prior to detecting the level of corin or portion thereof in the sample obtained from the individual.

50. A method of assessing whether an individual is at risk of developing congestive heart failure (CHF) comprising detecting the level of corin or portion thereof in the individual and comparing the level of corin or portion thereof in a sample of the individual to the level of corin or portion thereof in a normal control, or a baseline level of corin or portion thereof in the individual, wherein a decrease in the level of corin or portion thereof in the sample compared to the level of corin or portion thereof in the control or the baseline level is an indication that the individual is at risk of developing CHF.

51. The method of claim 50 wherein the level of corin or portion thereof in the individual is detected in a sample obtained from the individual.

52. The method of claim 50 wherein the sample is a body fluid.

53. The method of claim 52 wherein the body fluid is blood, plasma or a combination thereof.

54. The method of claim 50 wherein the corin is soluble corin.

55. The method of claim 50 wherein the individual is human.

56. The method of claim 50 wherein the control comprises one or more samples from one or more healthy individuals, a reference standard or a combination thereof.

57. The method of claim 50 wherein the level of a corin polypeptide or portion thereof is detected.

58. The method of claim 57 wherein the corin polypeptide or portion thereof is detected using an antibody having binding specificity for corin or a portion thereof.

59. The method of claim 58 wherein the antibody is a polyclonal antibody or a monoclonal antibody.

60. The method of claim 59 wherein the antibody has binding specificity for all or a portion of the extracellular domain of corin.

61. The method of claim 50 further comprising detecting the level of corin or portion thereof in the individual prior to comparing the level of corin or portion thereof in a sample of the individual to the level of corin or portion thereof in a control.

62. The method of claim 61 further comprising obtaining a sample from the individual prior to detecting the level of corin or portion thereof in the sample obtained from the individual.
